(19) United States
(12) Patent Application Publication
(10) Pub. No.: US 2006/0155315 A1
(43) Pub. Date: Jul. 13, 2006

(54) Cutting apparatus

(76) Inventor: Philip Crosland, Leicestershire (GB)

Correspondence Address: SMITH-HILL AND BEDELL, P.C., 16100 NW CORNELL ROAD, SUITE 220, BEAVERTON, OR, 97006, US

(21) Appl. No.: 11/262,040

(22) Filed: Oct. 28, 2005

(30) Foreign Application Priority Data
Oct. 30, 2004 (GB) 0424093.3

(51) Int. Cl. A61B 17/32

(52) U.S. Cl. 606/167

(57) ABSTRACT

Cutting apparatus is provided for cutting an aperture in an article. The apparatus comprises input means for inputting data relating to a profile of the aperture and a data processing arrangement for receiving said data and processing said data to provide instructions for operating a cutting assembly to cut the aperture into the article. The apparatus also includes a cutting assembly operable to move in accordance with instructions to cut the article.

This invention relates to cutting apparatus and to methods of cutting apertures in articles. In particular, but not exclusively, the invention relates to cutting apparatus for cutting apertures in flanges of stoma appliances and to methods for cutting apertures in flanges of stoma appliances.

In the stoma industry, it is important that the aperture in a flange of a stoma appliance has a shape and size which is the same as the shape and size of the profile of the stoma. The present way of cutting the flange of an appliance is by hand.

According to a first aspect of this invention, there is provided a method of cutting an aperture in an article, comprising inputting data relating to a profile of the aperture to be cut in the article into a data processing arrangement; processing the data to provide instructions for moving a cutting assembly to cut the aperture into the article; and operating the cutting assembly in accordance with said instructions to cut the article.

According to another aspect of this invention there is provided cutting apparatus for cutting an aperture in an article comprising: input means for inputting data relating to a profile of the apparatus; a data processing arrangement for receiving said data and processing said data to provide instructions for operating a cutting assembly to cut the aperture into the article; and a cutting assembly operable to move in accordance with said instructions to cut the article.

Preferably, the article comprises a stoma appliance, for example a stoma bag. The appliance may comprise a flange and the method is preferably for cutting the aforesaid aperture into a flange of the stoma appliance.

Prior to the step of inputting data, the method may comprise a step of providing an image of the profile of the aperture. The step of providing the image of the profile of the aperture may comprise providing a template of the profile of an element to be received in the aperture. The element may comprise a stoma on a patient.

The step of providing the image may further comprise copying a profile onto a substrate. The substrate may comprise a sheet of paper. The step of copying the profile may comprise tracing the profile in the template on to the aforesaid substrate to provide the aforesaid image.

The apparatus may include a scanner to scan the image to input the data into the data processing arrangement. The step of inputting data may comprise scanning the image to provide the aforesaid data. Preferably, the data is stored by the processing arrangement. Preferably, the data is stored as a raster file.

Preferably, the stored data is vectorised to provide a vector file, whereby the profile is represented as a plurality of vectors, which may be connected to one another. Preferably, the stored data is converted to a production file, in which the aforesaid pathway is represented as a plurality of position vectors, each extending from an origin. The origin may represent an initial position of the cutting device.

The production file may be edited to provide an edited production file to arrange the origin substantially centrally in the profile.

The cutting assembly may comprise a cutting device and a clamping arrangement. The clamping arrangement may comprise rotation means on which the article can be mounted. The cutting device may comprise a cutting member, and the rotation means may define a hole through which the cutting member can move to cut the article. The rotation means may comprise a turntable, which may include an annular member defining the aforesaid hole. The annular member is preferably rotatable to rotate the article. The rotation means may be rotatable relative to the cutting member.

The clamping arrangement may comprise at least one clamping member to clamp the article on the rotation means. Preferably, the clamping member is an annular in configuration, and may correspond in size and shape with the annular member.

The cutting member may be movable along two mutually perpendicular axes. The axes of movement may comprise a first axis substantially orthogonal to the rotation means, and a second axis generally parallel to the rotation means.

In order to cut the article, the cutting member may be moved along said first axis to engage the article and then may be moved at least once along the second axis to cut the aperture into the article. There may be a plurality of movements along the second axis to cut the article to a predetermined shape of the profile of the aperture.

Information relating to the article may be inputted to the processing arrangement. The article may define a starter aperture arranged in the region of the aperture to be cut. The starter aperture is preferably smaller than the aperture to be cut.

A main dimension of the starter aperture may be inputted to the processing arrangement. The starter aperture may be a circle in which the main dimension is a diameter.

A plurality of locating members may be provided to locate respective articles on the cutting device. Each locating member may correspond to a respective article, for example where the article is a stoma appliance, each locating member may correspond to a respective size of flange on the article and/or to a respective size of starter aperture in the flange.

Preferably, the method involves selecting an appropriate locating member to locate the article on the cutting device. Preferably, the locating member is received through the starter aperture.

The locating member may even include an outwardly extending arm to engage the article. Where the article comprises a stoma appliance, the arm engages the flange. The cutting member may travel along to cut the aperture.

In accordance with a third aspect of the invention there is provided computer software which, when installed on a data processing arrangement associated with a cutting apparatus in accordance with a second aspect of the invention, is operable to perform a method in accordance with the first aspect of the invention.

An embodiment of the invention will now be described by way of example, with reference to the accompanying drawings in which:

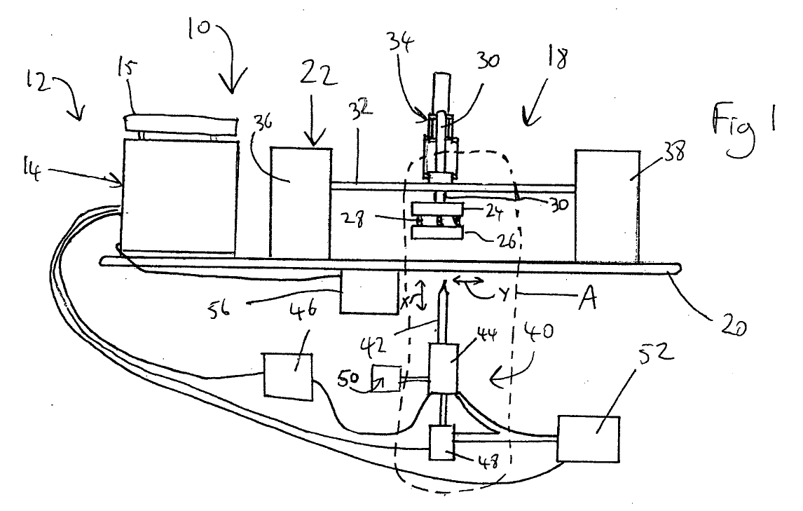

FIG. 1 is a diagrammatic front view of a cutting apparatus;

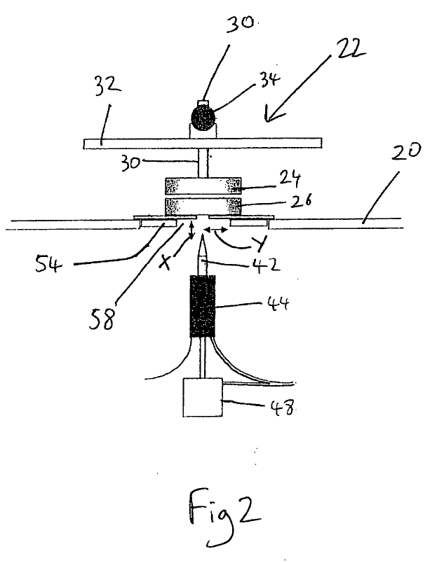

FIG. 2 is a sectional view of the region of FIG. 1 marked A;

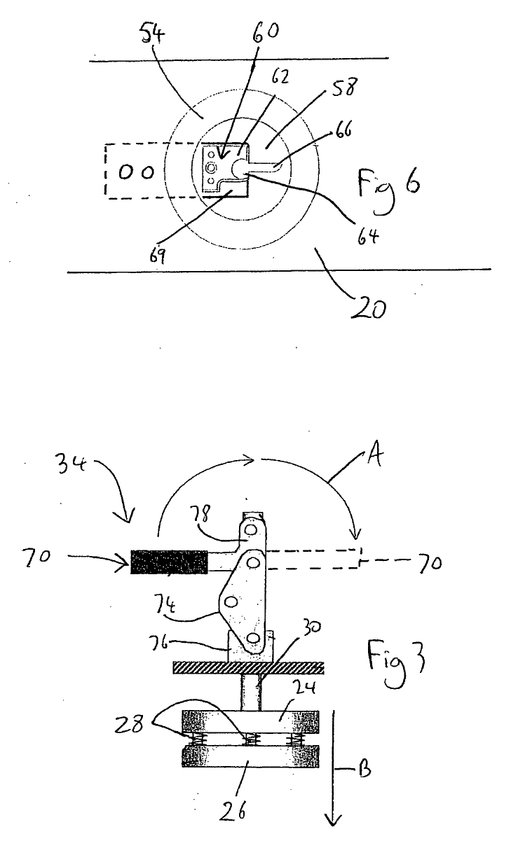

FIG. 3 is a side view of a clamping apparatus for use with the cutting apparatus shown in FIGS. 1 and 2;

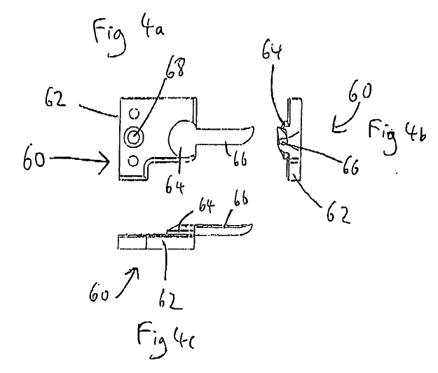

FIGS. 4a, 4b and 4c are respectively top end and side views of a locating member for use with the cutting apparatus shown in FIG. 1;

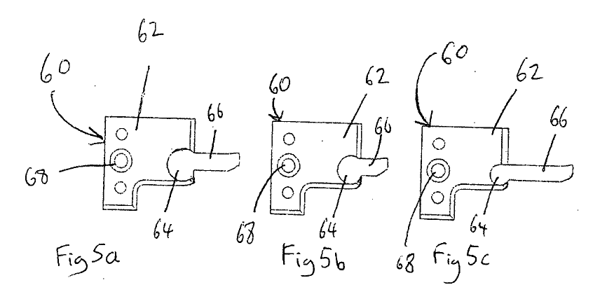

FIGS. 5a, 5b and 5c are top views of further locating members;

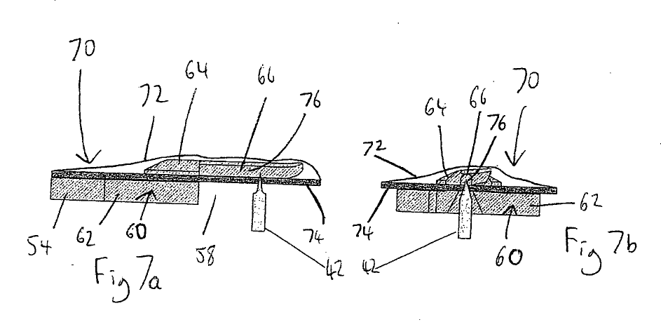

FIG. 6 is a top view of a locating member arranged in position; and

FIG. 7a shows a sectional side view of a locating member clamped in position; and FIG. 7b is a first view of the arrangement shown in FIG. 7a.

Referring to FIG. 1, there is shown a front view of cutting apparatus 10 for cutting an aperture into a flange of a stoma appliance. The cutting apparatus 10 comprises a control arrangement 12 in the form of a computer shown schematically at 14, which includes all the appropriate technical features of a computer, as would be appreciated by a person skilled in the art, including a data processing tower, a screen and a keyboard. A scanner 15 is provided in association with the computer for scanning images of stoma profiles, as will be explained below.

The apparatus 10 further includes a cutting assembly 18 comprising a cutting device 40 and a clamping arrangement 22 mounted on a bench 20.

The clamping arrangement 22 comprises aligned upper and lower clamping members 24, 26 connected to each other by a spring arrangement 28 (see FIG. 3) to enable two clamping members 24, 26 to be moved towards each other to a clamping position when clamping the flange of the stoma appliance and away from each other to a released position when released from the flange. A vertical shaft 30 extends upwardly from the clamping member 24 through a fixed horizontal planar element in the form of a shelf 32. A lever assembly 34 is connected to the shaft 30 to move the clamping members 24, 26 in to and out of the clamping position.

A pair of upwardly extending spaced support members 36, 38 are provided on the bench 20 to support the shelf 32, the clamping member 24, 26 and the lever assembly 34. The shelf 32 extends between the two support members 36, 38, with the clamping members 24, 26 and the lever assembly 34 being arranged on the shelf 32 between the support members 36, 38.

The cutting device 40 is arranged beneath the bench 20 and comprises a cutting member in the form of a knife 42 and an ultrasonic vibrator 44 to vibrate the knife at substantially 35 kHz. This allows the knife 42 to be maintained in a relatively blunt condition thereby improving safety.

The cutting device 40 also includes a piston 48 to move the knife 42 and the vibrating means 44 along the axis indicated by the double headed arrow X, i.e. directly upwardly and downwardly orthogonally relative to the bench 20.

The knife is connected to a generator 46 to activate the vibration means 44. The piston 48 is provided to move the knife 42 towards and away from the bench to engage the stoma appliance to be cut.

A displacement means 50 is provided to move the knife 42 in a straight line along the axis shown by the double headed arrow Y to cut the aperture into the stoma appliance. A compressor 52 supplies compressed air to the piston 48 and to the vibration means 44 to cool the components thereof.

Referring to FIG. 2, the cutting assembly 18 includes an annular turntable 54 against which the appliance is clamped by the clamping member 26. The turntable 54 defines an annular hole through which the knife 42 can extend. The turntable 54 is connected to a drive motor 56 (see FIG. 1) to rotate the turntable 54 about its axis. Controlled rotation of the turntable 54 coupled with controlled lateral movement of the knife 42 along the axis Y cuts the aperture into the flange so that the aperture has the correct profile.

The generator 46, the piston 48, the compressor 52 and the drive motor 56 are connected to the data processing arrangement 12 and are controlled thereby.

Referring to FIG. 4a to 4c and 5a to 5c, the apparatus further includes a plurality of locating members 60, each comprising a main body 62, a raised generally circular member 64 and an outwardly extending arm 66. The different locating members 60 have different sized raised members 64 to fit the different sized starter apertures of the flanges of different stoma appliances. Similarly, the different locating members 60 have different length arms 66 which correspond to the different radii of the flanges of different stoma appliances. Top, side and front views of one locating member 60 are shown in FIGS. 4a, 4b and 4c, and to views of a selection of other locating members 60 are shown in FIGS. 5a to 5c. It will be appreciated that the locating members shown in FIGS. 4a to 4c and 5a to 5c are only a selection of the locating members that can be used. Other configurations can be used.

The main body 62 of each of the locating members 60 shown in FIGS. 4a to 4c and 5a to 5c, is provided with apertures 68 to enable the main body 62 to be secured by bolts to the bench 20 via a tab 69 (see FIG. 6) which extends from the bench 20 to a region generally centrally of the hole 58 in the centre of the turntable 54. The raised circular member 64 and the arm 66 protrude above the upper surface of the turntable 54, and the appliance is arranged such that the arm 66 extends through the starter hole of the flange, and the starter hole is then arranged over the main central circular member 64. The raised circular member 64 has a size such that it is a close fit within the starter hole of the appliance flange. Similarly, the arm 66 is of such a length that it extends a sufficient distance to cover the horizontal movement of the knife 42.

When the stoma appliance is arranged over the locating member 60, the clamping arrangement 22 is then operated. The clamping arrangement 22 is shown in FIG. 3 and comprises the upper and lower clamping members 24, 26 which are connected by spring members 28. The shaft 30 extends upwardly from the upper clamping member 24 through the bench 20 to the lever assembly 34. The lever assembly 34 comprises a handle 70 and a lever linkage 72.

The lever linkage 72 comprises a first lever 74 pivotally mounted to a fixed bush 76. The fixed bush 76 is fixedly mounted on the bench 20. The opposite end of the lever 74 is pivotally attached to a second lever 78 which in turn is pivotally mounted to the shaft 30. The second lever 78 is fixedly mounted to the handle 70. By moving the handle in the direction indicated by the arrow A, the shaft 30, and the clamping members 24, 26 are moved downwardly as indicated by the arrow B. When the handle reaches the position shown in broken lines in FIG. 3, the lower clamping member 26 has engaged with the appliance, and the handle 30 is locked in place.

When the clamping assembly 22 is locked in place, the computer then activates the cutting device to move the knife 42 upwardly into engagement with the flange of the stoma appliance, and activates the generator 46 to cause the vibration means 44 to vibrate the knife 42 to cut the flange. The knife moves backwards and forwards on the axis Y as the drive motor 56 causes the turntable 54 and the flange on the bag to rotate so that an aperture is cut in the flange in accordance with a profile of a stoma which has been inputted in to the computer, as will be explained below. During the cutting operation, the arm acts as a guide for the knife 42 and to hold the back of the bag away from the knife 42 to prevent the bag being cut.

Referring to FIGS. 7a and 7b, there is shown a locating member 60 arranged on the turntable 54, with a stoma appliance 70 mounted thereon.

As can be seen, the stoma appliance 70 comprises a bag portion 72 and a flange 74, as would be appreciated by persons skilled in the art.

The outwardly extending arm 66 is shown in section and, as can be seen defines a groove 76 to receive the tip of the knife 42 to cut the flange 74. The tip of the knife 42 can travel along the groove 76 to effect the cutting of the desired profile of the aperture into the flange 74.

The procedure for inputting the data relating to the profile of the aperture to be cut is as follows.

A template of a patient's stoma is provided by a stoma care nurse. The template defines the profile of the stoma. This profile is traced from the template on to a clean sheet of paper. The sheet of paper is then placed into the scanner 15 and the traced profile is then scanned using scanning software, such as Scan2CAD V6 software with standard off the shelf scanning software to provide a scanned image in the form of a raster file. The scanned image is then edited by the user so that the profile is stored in the computer as a clean single line. This edited image is then vectorised by the software so that the raster image is represented as a vector file. The profile is thus represented as a plurality of connected vectors. The vector file is then converted to a production file that represents the profile as a plurality of position vectors relative to an origin. This origin represents the initial (rest) position of the tip of the knife 42. The production file is then edited so that the tip of each vector spaced from the origin is represented as coordinates on a graph. The origin is arranged in the centre of this profile.

The type of appliance which is used by the patient is then assigned to the production file. The patient's appliance will have a predetermined size of flange and starter hole and the size of the flange and starter hole are also assigned to the production file.

The production file then determines which of the locating members 60 is required to fit the size of flange and the starter aperture in the flange.

In the event that the aperture cut in the flange is a circle, a production file is created which comprises a plurality of position vectors each extending from an origin, and each being the same length. The position vectors thereby defining a circle having a radius the length of the vectors. The type of appliance and the size of the starter hole is then assigned to the file and the file is treated in the same way in which the file has been scanned in.

There is thus ascribed an apparatus in a flange of a stoma appliance which is efficient and easy to use and avoids the problems of the prior art.

Various modifications can be made without the departing from the scope of the invention, for example, a different type of cutting device could be used.

Whilst endeavouring in the foregoing specification to draw attention to those features of the invention believed to be of particular importance it should be understood that the Applicant claims protection in respect of any patentable feature or combination of features herein before referred to and/or shown in the drawings whether or not particular emphasis has been placed thereon.

1. A method of cutting an aperture in an article, comprising inputting data relating to a profile of the aperture to be cut in the article into a data processing arrangement; processing the data to provide instructions for moving a cutting assembly to cut the aperture into the article; and operating the cutting assembly in accordance with said instructions to cut the article.

2. A method according to claim 1 wherein the article comprises a stoma appliance, the appliance comprising a flange and the method comprises cutting the aforesaid aperture into a flange of the stoma appliance.

3. A method according to claim 1 wherein prior to the step of inputting data, the method comprises a step of providing an image of the profile of the aperture.

4. A method according to claim 3 wherein the step of providing the image of the profile of the aperture comprises providing a template of the profile of an element to be received in the aperture.

5. A method according to claim 3 wherein the step of providing the image further comprises copying the profile onto a substrate.

6. A method according to claim 5 wherein the step of copying the profile comprises tracing the profile in the template on to the aforesaid substrate to provide the aforesaid image.

7. A method according to claim 1 wherein the step of inputting data comprises scanning the image to provide the aforesaid data, the data being stored by the processing arrangement.

8. A method according to claim 7 wherein the data is stored as a raster file.

9. A method according to claim 7 wherein the stored data is vectorised to provide a vector file, whereby the profile is represented as a plurality of vectors, connected to one another.

10. A method according to claim 9 wherein the stored data is converted to a production file, in which the aforesaid profile is represented as a plurality of position vectors, each extending from an origin, the origin representing an initial position of the cutting device.

11. A method according to claim 10 wherein the production file is edited to provide an edited production file to arrange the origin substantially centrally in the profile.

12. A method according to claim 1 wherein the cutting assembly comprises a cutting device and a clamping arrangement, the clamping arrangement comprising rotation means on which the article can be mounted.

13. A method according to claim 12 wherein the cutting device comprises a cutting member, and the rotation means defines a hole through which the cutting member can move to cut the article.

14. A method according to claim 12 wherein the rotation means comprises a turntable, which includes an annular member defining the aforesaid hole.

15. A method according to claim 14 wherein the annular member is rotatable to rotate the article.

16. A method according to claim 12 wherein the rotation means is rotatable relative to the cutting member.

17. A method according to claim 12 wherein the clamping arrangement comprises at least one clamping member to clamp the article on the rotation means, the clamping member being annular in configuration, and corresponding in size and shape with the annular member.

18. A method according to claim 13 wherein the cutting member is movable along two mutually perpendicular axes, the axes of movement comprising a first axis substantially orthogonal to the rotation means, and a second axis generally parallel to the rotation means.

19. A method according to claim 18 wherein in order to cut the article, the cutting member is moved along said first axis to engage the article and is then moved at least once along the second axis to cut the aperture into the article.

20. A method according to claim 19 including a plurality of movements of the cutting member along the second axis to cut the article to a predetermined shape of the profile of the aperture.

21. A method according to claim 1 wherein information relating to the article is inputted to the processing arrangement, the article defining a starter aperture arranged in the region of the aperture to be cut, the starter aperture being smaller than the aperture to be cut.

22. A method according to claim 21 wherein a main dimension of the starter aperture is inputted to the processing arrangement.

23. A method according to claim 22 wherein the starter aperture is a circle in which the main dimension is a diameter.

24. A method according to claim 1 wherein a plurality of locating members are provided to locate respective articles on the cutting device, each locating member corresponding to a respective article.

25. A method according to claim 24 wherein where the article is a stoma appliance, each locating member corresponds to a respective size of flange on the article and/or to a respective size of starter aperture in the flange.

26. A method according to claim 25 wherein the method involves selecting an appropriate locating member to locate the article on the cutting device, the locating member being received through the starter aperture.

27. A method according to claim 25 wherein the locating member includes an outwardly extending arm to engage the article.

28. A method according to claim 27 wherein where the article comprises a stoma appliance, the arm engages the flange.

29. Cutting apparatus for cutting an aperture in an article comprising: input means for inputting data relating to a profile of the aperture; a data processing arrangement for receiving said data and processing said data to provide instructions for operating a cutting assembly to cut the aperture into the article; and a cutting assembly operable to move in accordance with said instructions to cut the article.

30. Cutting apparatus according to claim 29 wherein the apparatus includes a scanner to scan the image to input the data into the data processing arrangement.

31. Cutting apparatus according to claim 29 wherein the cutting assembly comprises a cutting device and a clamping arrangement, the clamping arrangement comprising rotation means on which the article can be mounted.

32. Cutting apparatus according to claim 31 wherein the cutting device may comprise a cutting member, and the rotation means may define a hole through which the cutting member can move to cut the article.

33. Cutting apparatus according to claim 32 wherein the rotation means comprises a turntable, which includes an annular member defining the aforesaid hole. The annular member being rotatable to rotate the article, and the rotation means being rotatable relative to the cutting member.

34. Cutting apparatus according to claim 31 wherein the clamping arrangement comprises at least one clamping member to clamp the article on the rotation means, the clamping member being annular in configuration, and corresponding in size and shape with the annular member.

35. Cutting apparatus according to claim 32 wherein the cutting member is movable along two mutually perpendicular axes, the axes of movement comprising a first axis substantially orthogonal to the rotation means, and a second axis generally parallel to the rotation means.

36. Cutting apparatus according to claim 31 wherein a plurality of locating members are provided to locate respective articles on the cutting device, each locating member corresponding to a respective article.

37. Cutting apparatus according to claim 36 wherein where the article is a stoma appliance, each locating member corresponds to a respective size of flange on the article and/or to a respective size of starter aperture in the flange.

38. Cutting apparatus according to claim 36 the locating member includes an outwardly extending arm to engage the article.

39. Computer software which, when installed on a data processing arrangement associated with a cutting apparatus comprising: input means for inputting data relating to a profile of an aperture; a data processing arrangement for receiving said data and processing said data to provide instructions for operating a cutting assembly to cut the aperture into the article; and a cutting assembly operable to move in accordance with said instructions to cut the article, is operable to perform a method that comprises inputting data relating to a profile of the aperture to be cut in the article into the data processing arrangement; processing the data to provide instructions for moving a cutting assembly to cut the aperture into the article; and operating the cutting assembly in accordance with said instructions to cut the article.
